(19) United States
(12) Patent Application Publication
(10) Pub. No.: US 2013/0133245 A1
(43) Pub. Date: May 30, 2013

(54) OXYGENATE ADDITIVE FOR INTERNAL COMBUSTION ENGINE FUEL, FUEL COMPOSITION COMPRISING THE SAME AND IMPROVED PROCESS FOR PRODUCING HIGHLY METHYLATED GLYCEROL ETHERS

(71) Applicants: Jyh-Shyong CHANG, Taipei City (TW); Tzong-Rong Ling, Taoyuan County (TW); Tse-Chuan Chou, Tainan (TW); Lawrence Chao-Shan Chou, Tainan (TW)

(72) Inventors: Jyh-Shyong CHANG, Taipei City (TW); Tzong-Rong Ling, Taoyuan County (TW); Tse-Chuan Chou, Tainan (TW); Lawrence Chao-Shan Chou, Tainan (TW)

(21) Appl. No.: 13/304,860

(22) Filed: Nov. 28, 2011

(51) Int. Cl. C10L 1/185; C07C 41/00

(52) U.S. Cl. 44/446; 568/671

(57) ABSTRACT

The invention relates to an oxygenate additive for an internal combustion engine fuel, comprising glycerol dimethyl ethers (GDMEs) and glycerol trimethyl ether (GTME); a fuel composition comprising the oxygenate additive; an improved process for methylation of glycerol with dimethyl sulfate to produce highly methylated glycerol ethers; and uses of ethers produced from the process for diesel fuel, for gasoline and for aviation turbine fuel, or as a green solvent or as an antifreezer.

### TECHNICAL FIELD

The invention relates to an oxygenate additive for an internal combustion engine fuel, comprising glycerol dimethyl ethers (GDMEs) and glycerol trimethyl ether (GTME); a fuel composition comprising the oxygenate additive; an improved process for methylation of glycerol with dimethyl sulfate to produce highly methylated glycerol ethers; and uses of ethers produced from the process for diesel fuel, for gasoline and for aviation turbine fuel, or as a green solvent or as an antifreezer.

### BACKGROUND OF THE INVENTION

Oxygenates can be added to fuels, such as diesel fuel, in order to reduce harmful exhaust emissions like particulate matters (PM), and, sometimes, NOₓ. The use of glycerol to synthesize oxygenates is commercially-promising, due to the overproduction of glycerol nowadays. Glycerol itself cannot be burned directly or be added to fuel oils since glycerol is hydrophilic and proceeds with polymerization at elevated temperatures thereby clogging engines and reducing the speed of engines, or part of the glycerol is oxidized to noxious acrolein, which is released to the atmosphere. However, glycerol can be used to synthesize glycerol alkyl ethers. The synthesized glycerol alkyl ethers are valuable oxygenate additives for diesel fuel since they can increase combustion efficiency and enhance the cetane number. For example, as disclosed in U.S. Pat. No. 5,308,365, glycerol tertiary butyl ether (GTBE) is a good oxygenate additive for diesel and biodiesel. In particular, glycerol tertiary butyl ether, when incorporated in standard 30-40% aromatic containing diesel fuel, provides reduced emissions of particulate matter, hydrocarbons, carbon monoxide, etc.

GTBE, as disclosed in U.S. Pat. No. 5,476,971, can be prepared by a process comprising reacting isobutylene with glycerol in the presence of an acidic catalyst, wherein the reaction mixture is phase-separated into a heavier glycerol and catalyst-containing polar phase which is conveniently recycled, and a lighter hydrocarbon phase from which product ethers can be readily separated.

In a conventional technique, glycerol alkyl ethers can be prepared by etherifying glycerol with alkenes or alcohols in the presence of an acidic catalyst at atmospheric pressure or high pressure. The prepared glycerol alkyl ethers include mono-, di-, tri-butyl ethers of glycerol. In view of the production of water from the reaction of glycerol with an alcohol, the removal of water during the processing (see US patent 20070238905) can increase the etherification degree of glycerol. When the etherification degree of glycerol is higher, the combustion efficiency thereof is higher, and the resulting product therefore is suitable for use as an oxygenate for fuels.

The etherification of glycerol with methanol produces glycerol mono-methyl ethers (GMMEs), glycerol di-methyl ethers (GDMEs), and glycerol tri-methyl ether (GTME), as shown in FIG. 1. Generally, highly methylated glycerol ethers include glycerol dimethyl ethers and glycerol tri-methyl ether, which can be burned directly or be used as an oxygenate additive for diesel fuel. However, possible oxygenate additives composed of GDMEs and GTME for diesel fuel, gasoline and aviation turbine fuel have not yet been developed.

Accordingly, the objectives of the invention are to provide an oxygenate additives for an internal combustion engine fuel, comprising GDMEs and GTME, wherein the internal combustion engine fuel is selected from the group consisting of: diesel fuel, gasoline and aviation turbine fuel; a fuel composition comprising the oxygenate additive and the internal combustion engine fuel, and in addition, to provide an improved process for methylation of glycerol with dimethyl sulfate to produce highly methylated glycerol ethers. The process has the advantages of high yield of highly methylated glycerol ethers but using reduced quantity of dimethyl sulfate, etc.

### SUMMARY OF THE INVENTION

The invention provides an oxygenate additive for an internal combustion engine fuel, comprising glycerol dimethyl ethers (GDMEs) and glycerol trimethyl ether (GTME), wherein glycerol dimethyl ethers (GDMEs) is in an amount of 0 to 25 wt % and glycerol trimethyl ether (GTME) is in an amount of 100 to 75 wt %, based on the total weight of the oxygenate additive.

The invention further provides an improved process for methylation glycerol with dimethyl sulfate. Namely, in a process for methylation of glycerol with dimethyl sulfate, which comprises: a) mixing glycerol with an aqueous solution of alkali hydroxide to form a solution of glycerate; b) mixing the solution of glycerate with dimethyl sulfate in a reactor to form highly methylated glycerol ethers, and c) separating the highly methylated glycerol ethers from the resulting solution, wherein steps a) to c) are conducted at a temperature of 25-200° C., the invention provides an improvement which comprises: replacing the glycerol of step a) with a mixture obtained by reacting glycerol with methanol in the presence of an acid catalyst at a temperature of 50 to 300° C.

The invention additionally provides a fuel composition comprising the oxygenate additive and internal combustion engine fuel, and uses of ethers produced from the process of the invention for diesel fuel, for gasoline and for aviation turbine fuel, or as a green solvent or as an antifreezer.

### BRIEF DESCRIPTION OF FIGURE

FIG. 1 shows formulae of two isomers of glycerol mono-methyl ethers (GMMEs), two isomers of glycerol dimethyl ethers (GDMEs) and a glycerol trimethyl ether (GTME), wherein FIGS. 1a and 1b show two isomers of GMME, FIGS. 1c and 1d show two isomers of GDMEs, and FIG. 1e shows GTME.

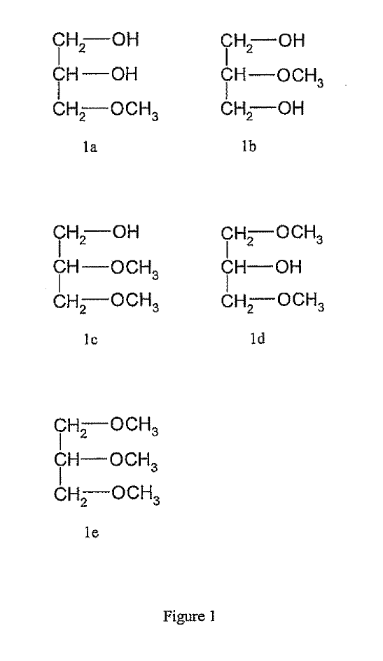

### DETAILED DISCLOSURE OF THE INVENTION

As used herein, the following definitions are applicable unless otherwise described.

The term “glycerol” used herein, also called “glycerine” has the formula:

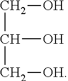

The term “aviation turbine fuel” used herein, also called “jet fuel” refers to a type of fuel designed for use in aircraft powered by gas-turbine engines.

The term “green solvent” used herein refers to a type of solvent, which when used in chemical production, can minimize the environmental impact.

Conventional process for methylation of glycerol with dimethyl sulfate, comprises:
a) mixing glycerol with an aqueous solution of alkali hydroxide of formula MOH, wherein M is a metal from group IA of the Periodic Table, to form a solution of glycerate, wherein an example of glycerate has the following formula:

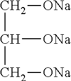

b) mixing the solution of glycerate with dimethyl sulfate in a reactor to form highly methylated glycerol ethers, and
c) separating the highly methylated glycerol ethers from the resulting solution, wherein steps a) to c) are conducted at a temperature of 25-200° C.,

The invention provides an improvement which comprises: replacing the glycerol of step a) with a mixture obtained by reacting glycerol with methanol in the presence of an acid catalyst at a temperature of 50 to 300° C., such that the needed amount of dimethyl sulfate, which is a hazardous substance, is reduced compared to the conventional process which is without involving the improvement of the invention.

Exemplary acid catalysts for use herein may be any suitable acid catalysts for etherification, and may include, but not limited to, sulfonic resins (such as Amberlyst 70, Amberlyst 15, Amberlyst 35 and Amberlyst 36), zeolites, sulfonated carbon catalysts, for example, prepared by sulphonation of the sugar char, prepared by pyrolyzing sugar (D-glucose) at 400° C. for 15 hours under nitrogen flow in a tube furnace, or combinations thereof. The highly methylated glycerol ethers produced by the process of the invention include GDMEs and GTME. The catalysts also include homogeneous catalyst such sulfuric acid, hydrogen chloride etc.

The use of oxygenate, such as ethers of glycerol, to produce relatively clean burning diesel fuels, has been noted for over fifty years. In recent years, tert-butyl ethers of glycerol with a high content of diethers produced by the etherification of glycerol with isobutylene or tert-butyl alcohol using homogeneous or solid acid catalysts have been considered promising as oxygenate additives for diesel fuels. However, the mixture of GDMEs and GTME as an oxygenate additive is even better than glycerol tertiary butyl ether, because of the much cheaper price of methanol as a feedstock to produce GDMEs and GTME than that of isobutylene or tert-butyl alcohol.

In this respect, the invention therefore provides oxygenate additives, comprising a mixture of GDMEs and GTME. The solubility of GTME in diesel fuel is very high to approach 100%. Also, GTME can be completely dissolved in gasoline or aviation turbine fuel. But, too much amount of GDMEs mixed with diesel fuel will bring about phase separation.

In an embodiment, the invention provides a fuel composition, comprising the oxygenate additive of the invention and an internal combustion engine fuel, wherein the oxygenate additive comprises glycerol dimethyl ethers (GDMEs) and glycerol trimethyl ether (GTME), and the amount of glycerol dimethyl ethers (GDMEs) in the fuel composition does not exceed 25 wt % based on the total weight of the fuel composition.

In another embodiment, the oxygenate additive of the invention can be mixed with the diesel fuel, such as petro-diesel fuel or biodiesel fuel in which the content of glycerol dimethyl ethers (GDMEs) in the mixture of the oxygenate additive of the invention and petro-diesel or biodiesel fuel does not exceed 25 weight %, wherein the mixture of the oxygenate additive of the invention and petro-diesel or biodiesel fuel includes: GDMEs, GTME, petro-diesel or biodiesel fuel. The purpose of adding the oxygenate additive of the invention in diesel fuel, is to reduce of harmful exhaust emissions like particulates (PM), and, sometimes, NOₓ.

In yet another embodiment, the oxygenate additive of the invention can be mixed with gasoline, in which the content of glycerol dimethyl ethers (GDMEs) in the mixture of the oxygenate additive of the invention and gasoline does not exceed 25 weight %, wherein the mixture of the oxygenate additive of the invention and gasoline includes: GDMEs, GTME, gasoline. The purpose of adding the oxygenate additive of the invention in gasoline is to enhance gasoline combustion and to reduce CO emission.

In yet another embodiment, the oxygenate additive of the invention can be mixed with aviation turbine fuel in which the content of glycerol dimethyl ethers (GDMEs) in the mixture of the oxygenate additive of the invention and aviation turbine fuel does not exceed 25 weight %, wherein the mixture of the oxygenate additive and aviation turbine fuel includes: GDMEs, GTME and aviation turbine fuel.

In still yet another embodiment, the ethers produced by the process of the invention, including GDMEs and GTME, can be used as an oxygenate additive for diesel fuel, for gasoline, for aviation turbine fuel, or can be used as a green solvent or as an anti freezer.

### EXAMPLE

Examples of the invention are given below by way of illustration, instead of limitation.

### Example 1

(A) In the first stage, glycerol was etherified with methanol in the presence of Amberlyst 70 catalyst in a high-pressure reactor.
   In the high-pressure reactor, 117 grams of glycerol and 163 grams of methanol were heated with 11.7 grams of Amberlyst 70 catalyst at 150° C. for 24 hours. The resulting products were analyzed by gas chromatography. The distribution of reactant mixture and product mixture of the etherification of glycerol was shown in Table 1.

TABLE 1
Distribution of reactant mixture and
product mixture of the first stage

| | Initial | Final | Final |
|---|---|---|---|
| Component | (g) | (g) | (Normalized, %) |
| Glycerol | 117 | 59.3 | 21.18 |
| Methanol | 163 | 33.6 | 12.00 |
| Dimethyl ether (DME) | | 59.1 | 21.11 |
| Glycerol mono-methyl ethers (GMMEs) | | 59.2 | 21.14 |
| glycerol dimethyl ethers (GDMEs) | | 9.7 | 3.46 |
| glycerol trimethyl ether (GTME) | | 0.0 | 0.00 |
| Water | | 48.2 | 17.21 |
| Unknown (to be identified) | | 10.9 | 3.89 |
| Total | 280.0 | 280.0 | 100.0 |

(B) 210 grams of product mixture obtained from the first stage was distilled at 115° C. to obtain an overhead product comprising 43.1 grams of water and 31.9 grams of methanol. The resulting 89.0 grams of bottom product, comprising 36.6 grams of glycerol, 36.5 grams of GMMEs, 6.0 grams of GDMEs, 6.7 grams of unknown and 3.2 grams of water, was used as a feed of the second-stage reaction.
   Second stage: methylation of glycerol, GMMEs and GDMEs at a normal atmospheric pressure
   84.4 grams of NaOH and 13.0 grams of water were added to 89.0 grams of bottom product obtained from the first stage, containing 36.6 grams of glycerol, 36.5 grams of GMME, 6.0 grams of GDMEs, 6.7 grams of unknown and 3.2 grams of water, in a tank along with agitation for 1 hour at 25° C. to obtain a solution of glycerate. The resulting solution of glycerate and 133.1 grams of dimethyl sulfate were added to a reactor and slowly mixed at 70° C. for 1 day. The resulting product mixture (highly methylated glycerol ethers) were analyzed by gas chromatography, with the result as shown in Table 2.

TABLE 2
Distribution of reactant mixture (glycerol, GMMEs
and GDMEs) and product mixture of the second stage

| | Initial | Final | Final |
|---|---|---|---|
| Component | (g) | (g) | (Normalized, %) |
| Glycerol | 36.6 | 0 | |
| NaOH | 84.4 | 0 | |
| Water | 16.2 | 36.1 | 11.54 |
| dimethyl sulfate | 133.1 | 0 | |
| Methanol | | 4.5 | 1.44 |
| GMMEs | 36.5 | 13.4 | 4.28 |
| GDMEs | 6.0 | 21.4 | 6.84 |
| GTME | | 49.6 | 15.86 |
| Unknown | 6.7 | | |
| Oil slurry (sodium sulfate, | | 187.8 | 60.04 |
| sodium hydroxide, dimethyl | | | |
| sulfate, water, methanol, | | | |
| GMMEs, GDMEs and GTME) | | | |
| Total | 312.8 | 312.8 | 100.00 |

(C) The product mixture obtained from the second stage was purified by the following steps: (1) separation of sodium sulfate solid by filtration, (2) distillation of the filtrate at 180° C. to obtain 88.0 grams of overhead liquid, (3) extraction of the liquid with 149.5 grams of chloroform, and (4) removal of chloroform by distilling the resulting extract phase at 100° C. 43.5 grams of bottom liquid obtained from step (4) contained GDMEs (22 wt %) and GTME (78 wt %).

### Example 2

(A) In the first stage, glycerol was etherified with methanol in the presence of Amberlyst 70 catalyst in a high-pressure reactor.
   In the high-pressure reactor, 117 grams of glycerol and 163 grams of methanol were heated with 113 grams of Amberlyst 70 catalyst at 190° C. for 24 hours. The resulting products were analyzed by gas chromatography. The distribution of reactant mixture and product mixture of the etherification of glycerol was shown in Table 3.

TABLE 3
Distribution of reactant mixture and
product mixture of the first stage

| | Initial | Final | Final |
|---|---|---|---|
| Component | (g) | (g) | (Normalized, %) |
| Glycerol | 117 | 18.3 | 6.54 |
| Methanol | 163 | 22.4 | 8.00 |
| Dimethyl ether (DME) | | 69.0 | 24.64 |
| Glycerol mono-methyl ether (GMMEs) | | 50.1 | 17.89 |
| Glycerol dimethyl ether (GDMEs) | | 29.7 | 10.61 |
| Glycerol trimethyl ether (GTME) | | 2.3 | 0.82 |
| Water | | 69.4 | 24.79 |
| Unknown (to be identified) | | 18.8 | 6.71 |
| Total | 280.0 | 280.0 | 100.0 |

(B) 210 grams of product mixture obtained from the first stage was distilled at 115° C. to obtain an overhead product comprising 63.5 grams of water, 21.5 grams of methanol and 2.3 grams of GTME. The resulting 107.5 grams of bottom product, comprising 16.1 grams of glycerol, 44.1 grams of GMMEs, 26.1 grams of GDMEs, 16.6 grams of unknown and 4.6 grams of water, was used as a feed of the second-stage reaction.
   Second stage: methylation of glycerol, GMMEs and GDMEs at a normal atmospheric pressure
   81.8 grams of NaOH and 11.1 grams of water were added to 107.5 grams of bottom product obtained from the first stag; containing 16.1 grams of glycerol, 44.1 grams of GMMEs, 26.1 grams of GDMEs, 16.6 grams of unknown and 4.6 grams of water, in a tank along with agitation for 1 hour at 25° C. to obtain a solution of glycerate. The resulting solution of glycerate and 128.9 grams of dimethyl sulfate were added to a reactor and slowly mixed at 70° C. for 1 day. The resulting product mixture (highly methylated glycerol ethers) were analyzed by gas chromatography, with the result as shown in Table 4.

TABLE 4
Distribution of reactant mixture (glycerol, GMMEs
and GDMEs) and product mixture of the second stage

| | Initial | Final | Final |
|---|---|---|---|
| Component | (g) | (g) | (Normalized, %) |
| Glycerol | 16.1 | 0 | 0 |
| NaOH | 81.8 | 0 | 0 |
| Water | 15.7 | 21.4 | 6.49 |
| dimethyl sulfate | 128.9 | 0 | 0 |
| Methanol | | 3.5 | 1.06 |
| GMMEs | 44.1 | 4.0 | 1.21 |
| GDMEs | 26.1 | 18.8 | 5.70 |
| GTME | | 24.9 | 7.55 |
| Unknown | 16.6 | | |
| Oil slurry (sodium sulfate, | | 257.2 | 77.99 |
| sodium hydroxide, dimethyl | | | |
| sulfate, water, methanol, | | | |
| GMMEs, GDMEs and GTME) | | | |
| Total | 329.8 | 329.8 | 100.00 |

(C) The product mixture obtained from the second stage was purified by the following steps: (1) separation of sodium sulfate solid by filtration, (2) distillation of the filtrate at 180° C. to obtain 63.5 grams of overhead liquid product, (3) extraction of the product (including GDMEs, GTME, etc.) with 151 grams of chloroform, and (4) removal of chloroform by distilling the resulting extract phase at 100° C. 35 grams of bottom liquid obtained from step (4) contained GDMEs (25 wt %) and GTME (75 wt %).

### Example 3

The laboratory of Chinese Petroleum Company (CPC) in Taiwan is currently analyzing the collected product of GDMEs (20 wt %) and GTME (80 wt %) mixture, which is manufactured via the methylation of glycerol with dimethyl sulfate in the presence of sodium hydroxide. The main properties of the new oxygenative mixture, which is composed of GDMEs (20 wt %) and GTME (80 wt %), are shown in Table 3, wherein DGM (diethylene glycol dimethyl ether) and DBE (dibutyl ether) are known as diesel cetane enhancers. From the comparison, we conclude that the product mixture of GDMEs (20 wt %) and GTME (80 wt %) can serve as a new oxygenate additive for diesels (biodiesel or petro-diesel).

TABLE 1
Comparison of main properties of the new oxygenate additive with the existing additives

| | Oxygenate mixture* | | | Existing Oxygenates | |
|---|---|---|---|---|---|
| | GDMEs | | GTME | DGM | DBE |
| CAS No. | 623-69-8 | 40453-77-8 | 20637-49-4 | 11-96-6 | 142-96-1 |
| Chemical formula | C₅H₁₂O₃ | C₅H₁₂O₃ | C₆H₁₄O₃ | C₆H₁₄O₃ | C₈H₁₈O |
| Chemical structure | (CH₃OCH₂)₂CHOH | | | (CH₃OCH₂CH₂)₂O | (C₄H₉)₂O |
| | CH₃OCH₂CH(OCH₃)CH₂OH | | | | |
| | | (CH₃OCH₂)₂CHOCH₃ | | | |
| Molecular weight, kg/kmol | 120 | 120 | 134 | 134 | 130.2 |
| Normal boiling point, K | 442 | 453 | 421 | 435 | 414 |
| Density, kg/m³ | | 968 | | 937 | 764 |
| Heat of combustion, J/kg | | −2.74 × 10⁷ | | −2.52 × 10⁷ | −3.80 × 10⁷ |
| Flash point, K | | 322 | | 343 | 298 |
| Lower flammability limit, % vol | | 1.47 | | 1.2 | 1.5 |
| Autoignition temperature, K | | — | | — | 467 |
| Cetane number | | 58 | | 112 | 91-100 |
| Lower heating value, kJ/g | | 25.12 | | 24.5 | — |

*The oxygenate mixture is composed of GDMEs (20 wt %) and GTME (80 wt %).

1. An oxygenate additive for an internal combustion engine fuel, comprising glycerol dimethyl ethers (GDMEs) and glycerol trimethyl ether (GTME), wherein glycerol dimethyl ethers (GDMEs) is in an amount of 0 to 25 wt % and glycerol trimethyl ether (GTME) is in an amount of 100 to 75 wt %, based on the total weight of the oxygenate additive.

2. The oxygenate additive according to claim 1, wherein the internal combustion engine fuel is selected from the group consisting of: diesel fuel, gasoline and aviation turbine fuel, preferably diesel fuel.

3. The oxygenate additive according to claim 2, wherein the diesel fuel is petro-diesel fuel or biodiesel fuel.

4. A fuel composition, comprising the oxygenate additive according to claim 1 and an internal combustion engine fuel, wherein the oxygenate additive comprises glycerol dimethyl ethers (GDMEs) and glycerol trimethyl ether (GTME), as defined in claim 1, and the amount of glycerol dimethyl ether (GDMEs) in the fuel composition does not exceed 25 wt % based on the total weight of the fuel composition.

5. A fuel composition according to claim 4, wherein the internal combustion engine fuel is selected from the group consisting of diesel fuel, gasoline and aviation turbine fuel, preferably diesel fuel.

6. A fuel composition according to claim 5, wherein the diesel fuel is petro-diesel fuel or biodiesel fuel.

7. A process for methylation of glycerol with dimethyl sulfate, which comprises: a) mixing glycerol with an aqueous solution of alkali hydroxide to form a solution of glycerate; b) mixing the solution of glycerate with dimethyl sulfate in a reactor to form highly methylated glycerol ethers, and c) separating the highly methylated glycerol ethers from the resulting solution, wherein steps a) to c) are conducted at a temperature of 25-200° C., wherein the improvement comprises: replacing the glycerol of step a) with a mixture obtained by reacting glycerol with methanol in the presence of an acid catalyst at a temperature of 50 to 300° C.

8. The process according to claim 7, wherein alkali hydroxide is selected from the group consisting of: LiOH, NaOH and KOH.

9. The process according to claim 7, wherein the acid catalyst is selected from the group consisting of a sulfonic resin, a zeolite and a sulfonated carbon catalyst.

10. The process according to claim 7, wherein the highly methylated glycerol ethers include glycerol dimethyl ethers (GDMEs) and glycerol trimethyl ether (GTME).

11. The process according to claim 7, wherein the produced ethers are used as an oxygenate additive for diesel fuel, for gasoline, for aviation turbine fuel, or as a green solvent or as an antifreezer.
